(19) United States
(12) Patent Application Publication
(10) Pub. No.: US 2020/0405557 A1
(43) Pub. Date: Dec. 31, 2020

(54) COMBINED PATIENT MEDICAL TRANSFER AND X-RAY APPARATUS

(71) Applicants: Leon Hochman, Farmington, MI (US); Nancy Valvona, Henrico, VA (US); Stephen Nicolato, Allen Park, MI (US)

(72) Inventors: Leon Hochman, Farmington, MI (US); Nancy Valvona, Henrico, VA (US); Stephen Nicolato, Allen Park, MI (US)

(21) Appl. No.: 16/843,000

(22) Filed: Apr. 8, 2020

Related U.S. Application Data
(60) Provisional application No. 62/831,935, filed on Apr. 10, 2019.

(51) Int. Cl. A61G 7/10; A61B 6/04

(52) U.S. Cl. CPC A61G 7/103; A61B 6/0442; A61G 2210/50; A61G 7/1028; A61G 7/1034

(57) ABSTRACT

A medical transfer system or apparatus including a patient transfer member including a lower support structure and a comb-like toothed sine wave apparatus for moving the transfer member and patient between a patient transporter table and a patient x-ray examining surface, each formed of a radiolucent material, wherein the toothed member is removed and the patient remains on the transport support during an x-ray procedure. The support structure in one embodiment defines a continuous sine wave, the toothed member including a series of transfer beams that fit into spaces formed in the support structure below the transfer member, and each transfer beam including a series of wheels that are actuated to move down and up whereby to lift or lower the patient transfer member.

### BACKGROUND OF THE INVENTION

### 1. Field of the Invention

The present invention pertains to a transporter table system for transporting and transferring a patient between a transporter table and a surgical table. More particularly the present invention concerns a medical transfer system or apparatus including a patient transfer member and a comb-like toothed apparatus for facilitating a patient x-ray while on a surgical table.

### 2. Description of the Prior Art

A transporter table system for rollably transporting and transferring a patient between two patient support surfaces is known, such as illustrated by U.S. Pat. No. 9,463,127, Transporter Table System, issued to Hochman et al., Oct. 11, 2016, the disclosure of which is hereby incorporated by reference. The Hochman system while useful did not address the issue of providing a patient support that could be used with and during CT and x-ray procedures.

Importantly, metal is contraindicated during x-ray and radiographic procedures and radiotranslucent materials are desired.

### OBJECTS OF THE INVENTION

An object according to this invention is provision of a medical bed and patient transfer system that is simple, compact, can be used in many areas, such as hospitals but also in clinics and doctor offices, similar medical examining facilities and activities, and an apparatus that is inexpensive and is capable of being adapted for assist in the transfer of patients in a variety of radiographic applications.

Another object of this invention is provision of a medical or examination bed or table with a simple patient raising and transfer apparatus which is space efficient and allows optimum use of small medical examination areas.

Advantageously, as a general object according to this invention, there is provision of an examination table or bed that is movable or stationary, and includes a patient lifting, raising, transfer and actuator apparatus.

It is to be appreciated that there is a need for an efficient low-cost medical bed or table system for patient transfer, such as described herein.

### SUMMARY OF THE INVENTION

In the first embodiment hereof, the present invention includes a patient transfer table and a surgical and/or x-ray table. A patient support member is transferable between the patient transfer member or table (gurney) and the surgical table. The patient support system includes a sinusoidal or comb-like structure and a mat disposed thereatop for supporting a patient thereon. The sinusoidal or comb-like member or pallet is formed from sufficiently strong material to support a patient thereon and is made from a radiolucent material to enable an x-ray of the patient to be taken when on the x-ray table without interfering with the clarity or quality of the x-ray.

According to this invention, in another aspect, there is provided a medical transfer system for moving a patient between a patient transport support and a patient x-ray examining surface, the transfer system comprising:

a patient transfer member, the transfer member comprising a patient support member having an upper surface for supporting a patient and a lower surface, and a series of supports extending between the upper and lower surfaces fixedly connected to said lower surface for supporting the transfer system and said patient transport support and examining surface, the supports in spaced apart relation between the opposite longitudinal ends of the surfaces, forming receiving spaces, and each support extending downwardly a like distance from the lower surface, the support member and supports comprised of a radiolucent material, and having a comb-like toothed apparatus disposed beneath a patient, the comb-like toothed apparatus extending transversely from an actuator bar in spaced apart relation, and transfer beams each adapted to fit into the spaces formed between respective of the spaced apart supports, each transfer beam includes a series of wheels that are actuated to simultaneously move down and up relative to the respective transfer beam whereby to lift or lower the patient from the operating table to the transport member or transfer member, and

wherein when the wheels are raised the transfer beams may enter or retract from the spaces formed by the comb-like member and when the transfer beams are lowered the wheels raise the transfer member into contact with and lift the transfer member and the actuator bar allow the transfer member with patient to be rolled to the opposite patient surface.

According to a preferred embodiment, the supports in spaced apart relation define successive portions of a sine wave, wherein successive upper peaks of the wave are connected to the lower surface and successive lower peaks of the wave form the supporting structure and which may be disposed on the surgical system or may be part of a component of the transport system and which is movable between the operating table and the transport table (gurney).

Further, the support structure that defines the sine wave is formed by a radiolucent material that differs in vertical thickness across the patient support member in that a vertically directed x-ray through the transfer member and support structure sees the same total thickness of material everywhere.

Preferably, the patient support member and supports produce substantially equal attenuation everywhere and no table induced artifacts on a radiograph.

The series of spaced apart supports of a radiolucent material are formed as one integral unit that varies in thickness and uniformly attenuates electromagnetic waves passing therethrough. The vertical integral unit describes a sine wave with peaks and valleys, respectively, fixedly connected to the lower surface and adapted to form a support for the transfer system, the thicken of the vertical integral unit being such that the thickness of the support unit at the peak and valley is about 50% greater than the vertical thickness of the support unit therebetween.

According to this invention, the medical transfer system is of a radiolucent material, meaning that it is virtually transparent to x-rays and appears black on x-ray images. While carbon fiber is generally preferred, the radiolucent material may also be selected from the group consisting of polymethylmethacrylate (PMMA), PETG co-polyester, and expanded polystyrene foam.

In an alternate preferred embodiment of a medical transfer system according to this invention, the series of supports comprises a series of separate inflatable feet of a suitable radiolucent material fixedly connected to the bottom surface off fabric, the material of minimal thickness for improved x-ray contrast.

In yet another alternate embodiment of a medical transfer system according to this invention, the series of supports comprises a series of separate and movable feet of a suitable radiolucent material connected to the bottom surface of the patient support, the feet location improving imaging contrast of x-rays through the patient support.

In a preferred arrangement, movement of the patient transfer system is achieved by raising or lowering the rollers or wheels on the support beams.

In a preferred arrangement, removable means lock the comb like toothed apparatus to the patient transfer member to enhance the toothed apparatus in moving the patient transfer member.

According to this invention, there is provided a medical table assembly, comprising a bed frame; a support structure mounted atop the frame for supporting a person in a plane of the frame, the frame and support defining an interior chamber, said support including an array of openings, a lifting rack mounted in the chamber for upward/downward vertical movement between the bed frame and the support structure, said lifting rack including an array of rollers for entry into respective of said openings, the rollers for supporting, lifting and moving a person when the lifting rack is raised and in the upward position, means for raising and lowering the lifting rack vertically upward and downward relative to the bed frame, wherein said means for raising and lowering comprises a selectively actuatable pneumatic air source, and at least one pneumatic actuator in fluid connection to the air source, the actuator having a piston that provides a selective alternating in and out displacement movement upon selective actuation of the air source to move the lifting rack and associated rollers relative to the support structure and the rollers relative to the opening as desired, wherein the pneumatic actuator is connected to one of the lifting rack and the support structure and the outward end of the piston thereof is adapted to engage the other of the lifting rack and the support structure.

In a preferred arrangement, the means for raising and lowering the lifting rack comprises an array of separate and spaced apart pneumatic actuators, each of the actuators in the array thereof connected in parallel to said air source to operate simultaneously to move the pistons in and out and raise and lower the lifting rack and associated rollers. However, electric and hydraulically-operated actuators may be used.

Preferably, said lifting rack includes a plurality of laterally spaced linear beams, some of said beams mounting and registering a selected number of the rollers in position for entry into respective of the openings and other of said beams for stiffening and providing rigidity to the lifting rack.

Preferably, said bed frame includes a plurality of laterally spaced linear spacer flanges, said flanges extending upwardly from said frame and into said interior in an amount sufficient to support and space the lifting rack above the pneumatic actuator, or array of actuators, upon lowering retraction of the lifting rack from the support structure.

In one arrangement, the pneumatic actuator is a single acting air valve, comprising a spring or bias member disposed internally of the actuator which constantly biases the piston thereof in one direction against the air pressure.

In another arrangement, the pneumatic actuator is a double acting air valve, comprising a pair or air inlets that selectively receive air from the pneumatic pump to force the piston thereof in opposite direction.

In one preferred arrangement, the pneumatic actuator, or actuators, is/are fixedly connected to the bed frame and the respective piston end, or ends, is/are fixedly connected to the lifting rack.

In this preferred embodiment, the rollers comprise a plurality of wheels that are adjusted in respective rectangular shaped openings.

In another preferred embodiment according to this invention, said patient support structure includes an array of generally round openings, and the lifting rack is characterized as a ball transfer table that is mounted in the chamber for upward/downward vertical movement by the actuators between the bed frame and the support structure, the lifting rack or ball transfer table including a fixed array of rolling ball assemblies that include a rolling ball sized for entry into respective of said round openings, the rolling balls for raising a person and rolling translating movement atop the support, such as for patient positioning atop the support structure, or movement between opposite ends, or in a side by side movement and transfer from the support table.

Additionally, the roller wheels or rolling balls may be other, such as rolling caster wheels that that are connected by stema to the lifting rack and the wheels thereof raised and lowered into the openings of the support structure and allow 360° rotation relative to their connections to the lifting rack and support openings to move the patient into a desired position.

The present invention will be more clearly understood with reference to the accompanying drawings and to the following Detailed Description, in which like reference numerals refer to like parts and where:

### BRIEF DESCRIPTION OF THE DRAWINGS

FIGS. 1-3 are elevation views, in perspective, looking at the stages of a patient transfer system or apparatus having a radiolucent structure being readied to move a patient from a transporter or gurney (FIG. 1), from the gurney (FIG. 2), and onto an x-ray table (FIG. 3) for examining the patient, a comb like toothed apparatus or a continuous sine wave shaped support structure attached to a bottom surface of the patient support member according to this invention;

FIGS. 4 and 4a are enlarged partial side views of the patient support transfer system being moved from the x-ray table and back onto the gurney;

FIGS. 4b and 4c are elevational views showing the elevating and lowering of the pallet;

FIG. 4d illustrates how the wheel assembly works without the cover for simplicity

FIG. 4e is a graph showing the x-ray effect of a radiolucent pallet used herein;

FIGS. 5a and 6b depict an alternate embodiment according to this invention wherein separate inflatable supports of radiolucent material are in side by side relation at selected locations of a radiolucent support member to support the patient on the gurney or x-ray table;

FIGS. 6a and 6b show an alternate embodiment according to this invention wherein a series of separate and movable feet of squared cross-section and of a suitable radiolucent material are fixed at selected locations support the patient support member on the gurney or x-ray table, the selected feet locations improving imaging contrast of x-rays through the patient support;

FIG. 7 is a perspective view showing a patient support disposed between a surgical table and a transfer table (gurney);

FIG. 8 is an exploded view showing components of the medical bed and transfer arrangement of FIG. 1 including a patient support, an adjustable lift structure, and rollers or wheels to support and transfer a patient according to a preferred embodiment of this invention;

FIG. 9 is another preferred embodiment of this invention and an exploded view showing the medical bed of FIG. 1 wherein the support structure is modified to include an array of round openings and the lifting rack is modified to include an array rolling ball assemblies wherein respective balls enter into respective of said round openings for patient transfer and positioning in multiple directions atop the support structure;

FIG. 10 is a view of rolling ball assembly and rolling ball;

FIG. 11 illustrates a modification wherein the rolling ball assembly is replaced with a conventional caster wheel with connecting stem and a wheel sized to appropriately enter round openings of the lifting rack and rotate as desired to position and transfer a patient atop the support.

### DETAILED DESCRIPTION OF THE PREFERRED EMBODIMENT

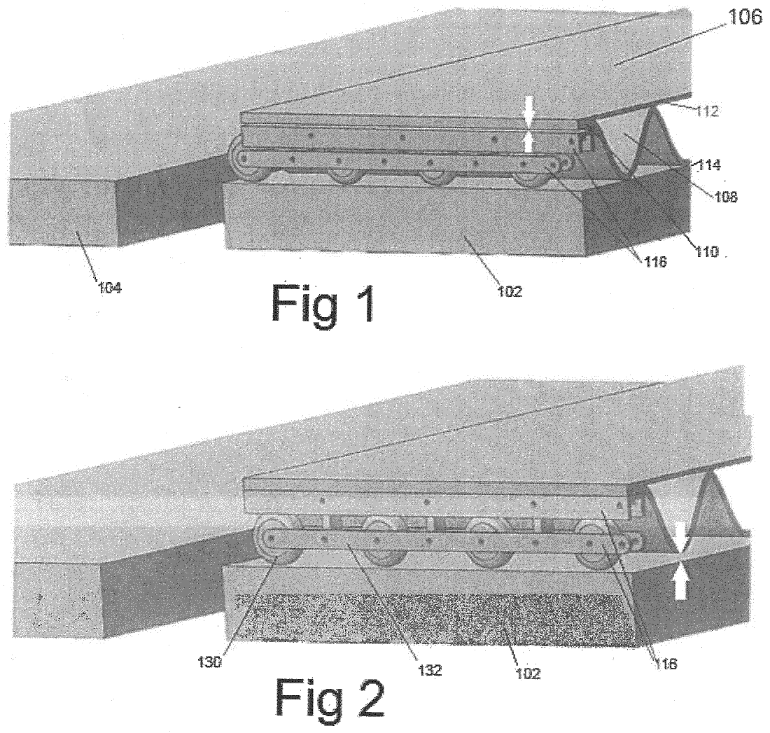

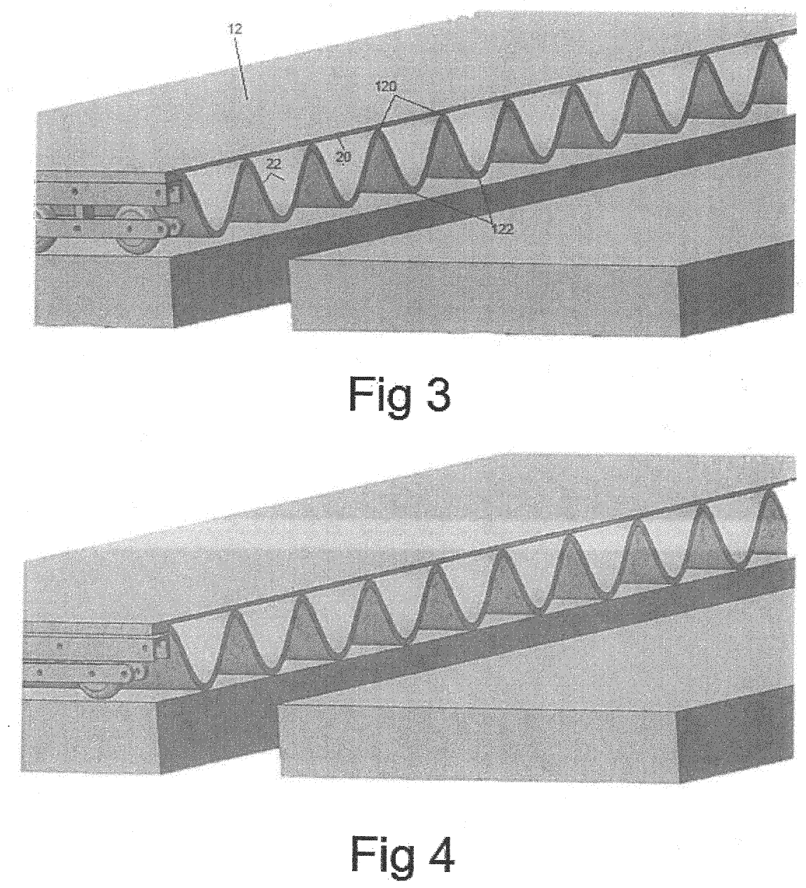

Turning now to the drawings, FIGS. 1-4 illustrate the medical transfer system 100 for moving a patient between a patient transport support “GURNEY” 102, such as a transportable gurney, and a patient x-ray examining surface “X-RAY” 104.

The medical transfer system or apparatus 100 comprises a patient transfer member 106 as described below. Typically, the transfer system member 106 is set atop the gurney 102, with the patient placed thereatop. The gurney is moved into juxtaposed abutment with the surgical/x-ray table 104, and the patient is transferred onto the surgical/x-ray table system 104, and the user pulls/rolls a conjoined toothed apparatus 108 and the transfer system apparatus 100 and patient thereon is transported from the gurney onto an operating table or x-ray table, whereupon the toothed apparatus is disposed beneath the patient and an x-ray procedure initiated. Thereafter, the procedure is reversed.

The conjoined tooth or sine wave apparatus 108 includes a thin, planar, generally rectangular shaped longitudinally extending patient support member 110 of a radiolucent material having an upper surface 112 for supporting a patient and a lower surface 114, and a series of supports 116 of radiolucent material fixedly connected to the lower surface for supporting the transfer system on one of the patient transport support GURNEY and examining surface X-RAY.

The supports 116 extend in spaced apart relation between the opposite longitudinal ends of the patient support member and extend in parallel relation between the opposite lateral sides of the support member to define a series of spaced apart spaces. The supports each extend vertically downward a like distance from the lower surface 20.

According to a preferred embodiment, the side by side supports 22 define a continuous sine wave, wherein successive upper peaks 120 of the wave are connected to the lower surface 20 and successive lower peaks 122 of the wave combine to define the supporting structure. Because of the nature of the support structure in the form of a sine wave, the patient transfer system or table 12 is reinforced as well as made compatible for radiographic purposes.

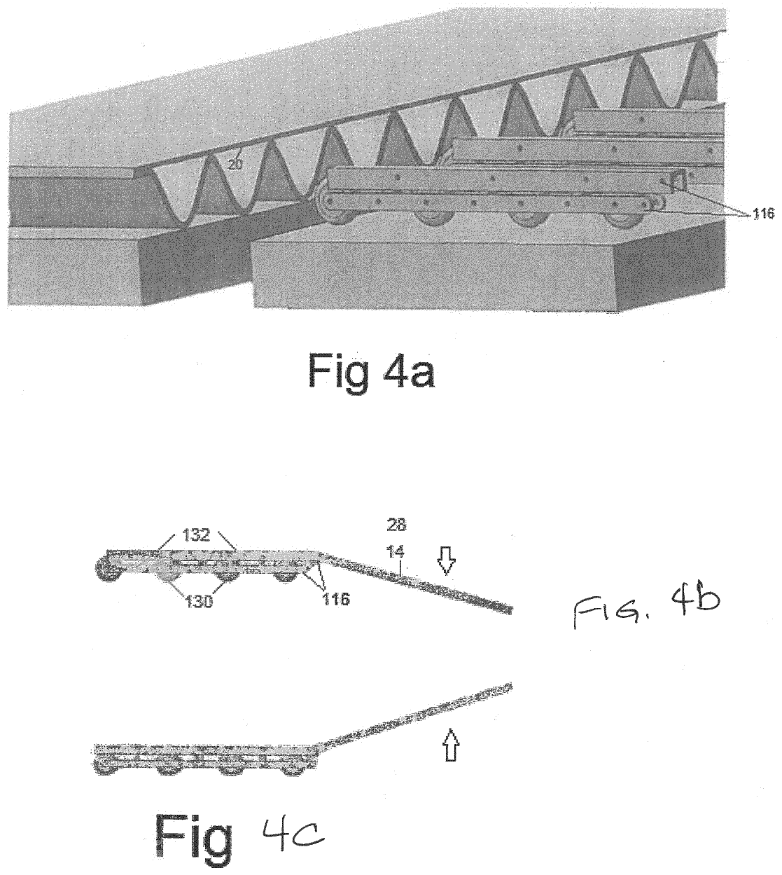

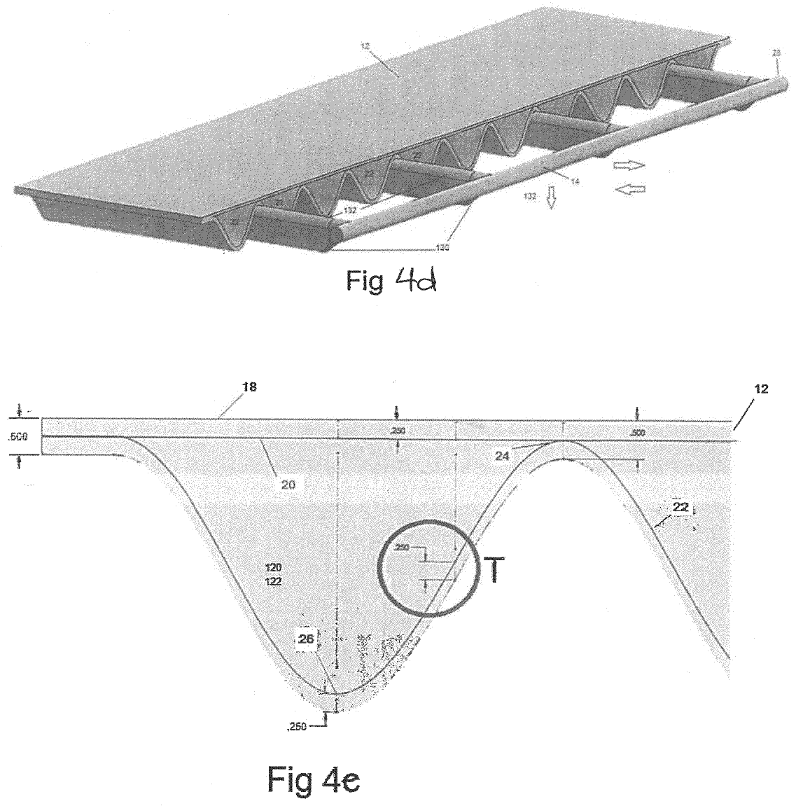

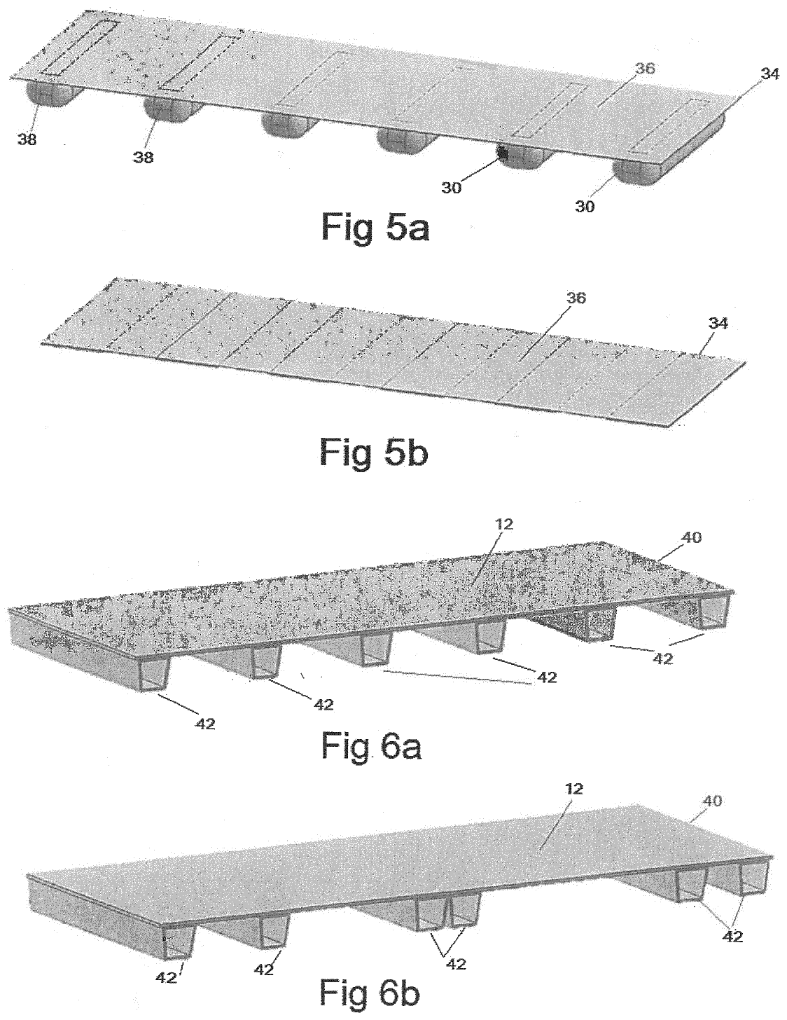

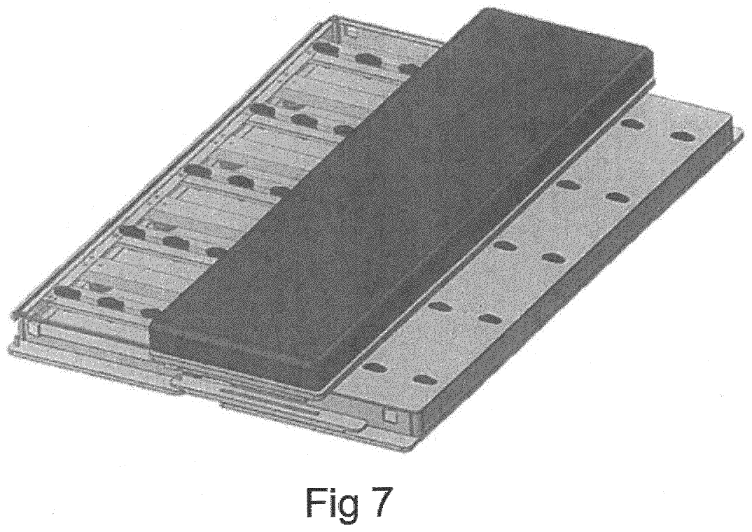

Referring to FIG. 4e, the support structure that defines the sine wave is formed by a radiolucent material that differs in vertical thickness across the patient support member in that a vertically directed x-ray through the transfer member and support structure sees the same total thickness of material everywhere. The vertical thickness of the material forming the support structure sine wave being such that the thickness of the support structure material at the peak and lower peak being about 50% greater than the vertical thickness “T” of the sine wave support material generally midway between the upper and lower peaks 24 and 26.

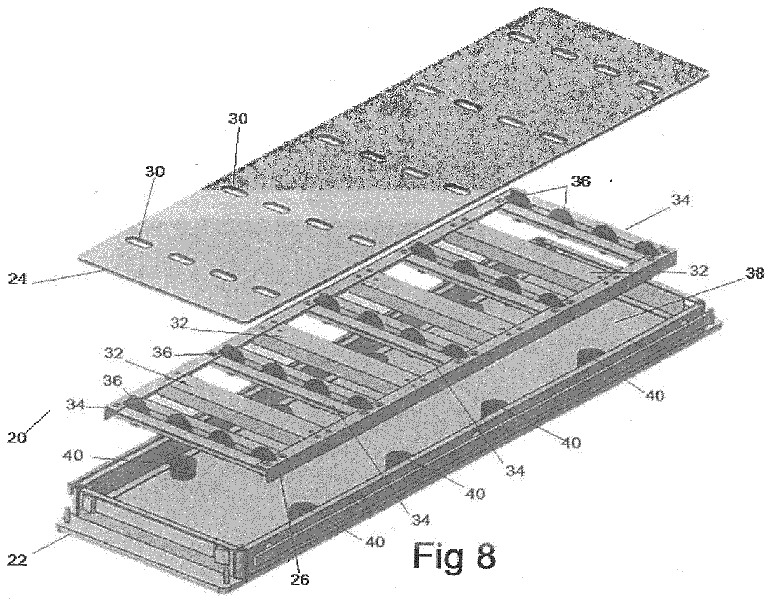

As shown in FIG. 8, preferably, the patient support member 12 and supports 22 produce substantially equal attenuation everywhere and no table induced artifacts on a radiograph.

Preferably, the support member and the supports are comprised of the radiolucent material, meaning that the material is virtually transparent to x-rays and appears black on x-ray images. While carbon fiber is generally preferred, the radiolucent material may be selected from the group consisting of carbon fiber, polymethylmethacrylate (PMMA), PETG co-polyester, and expanded polystyrene foam.

Referring to FIG. 1 through FIG. 4d, as shown, when the wheels 130 are lowered the transfer beams 132 do not engage the transfer member or support and may enter or leave the spaces formed by the supports 22. When the wheels 140 are raised, the transfer beams 30 are raised and engage the support 22 to raise the transfer member 12. The actuator 14 with raised wheels allows the raised transfer member with patient to be rolled to the surgical table patient surface. Thereafter, the wheels 32 are lowered.

The wheels are extended, lifting the pallet off the gurney.

The pallet is wheeled over to the table.

The wheels are retracted, lowering the pallet to the table.

The wheels are returned to the gurney.

As shown in the drawing, the means for raising and lowering the wheels comprises at least a pair and, preferably, a plurality of guide rails positioned intermittently on the platform.

The guide rails are secured to the pallet at least one on each end of the platform.

Each wheel is mounted for vertical movement by any suitable means such as electric, hydraulic or manual. Each guide rail has a plurality of wheels or rollers secured thereto.

As shown in FIG. 4c, the wheels 130 are secured to the rails by any suitable means, such as being journaled on shafts. Each shaft is connected to a lever of any suitable means, such as, for example, by being pinned or alternatively, the lever may have a fork at the end thereof with the camshaft being disposed in the open space between the two tines of the fork. A transverse pin projects through the tines and the shaft to connect the lever to the camshaft.

The lever can be rotated in an upward position and the rollers can be retracted from the interstices.

It should be appreciated that by rotating the lever downwardly this will, in turn, cause the wheels to lower. Clearly the reverse motion will cause the wheels to retract downwardly.

It is to be understood that when an array of rollers are projected into the interstices of the pallet, each with its own guide rail, and the gurney is brought into proximity with a surgical table by lifting the pallet via the rollers, this enables the pallet and the pad thereatop to be shifted onto the operating table.

As shown particularly in FIGS. 4b and 4d to transfer the patient back to the gurney the reverse procedure is used. Further, in a preferred arrangement, while not shown as conventional, removable means lock or otherwise may be used to interconnect the comb like toothed apparatus to the patient transfer member to enhance the ability of the toothed apparatus in moving and/or pulling the patient transfer member to and back from patient beds.

As shown in FIG. 8, transfer assemblies are each adapted to fit into respective of the spaces between respective of the spaced apart supports formed by the sine wave. Here, each of a plurality of transfer beams includes a series of wheels 32 and the wheels of the transfer beams are actuated simultaneously to move down and up relative to the respective transfer beam to which attached whereby to lift or lower the patient transfer member 12.

The support structure 24 for supporting the patient includes an array of openings 30. In the embodiment shown, the array includes five rows of four, generally, rectangular-shaped openings 20 or 30? in side by side relation. The number and size of the openings may be different, depending on the services to be provided and the patients to be supported or raised.

The lifting rack 26 is mounted in the chamber 38 for upward/downward vertical movement between the bed frame and the support structure. The lifting rack 26 includes an array of laterally spaced linear beams 32 and 34 that are disposed in parallel relation to one another. A first set of the beams 32 are for stiffening and providing rigidity to the lifting rack.

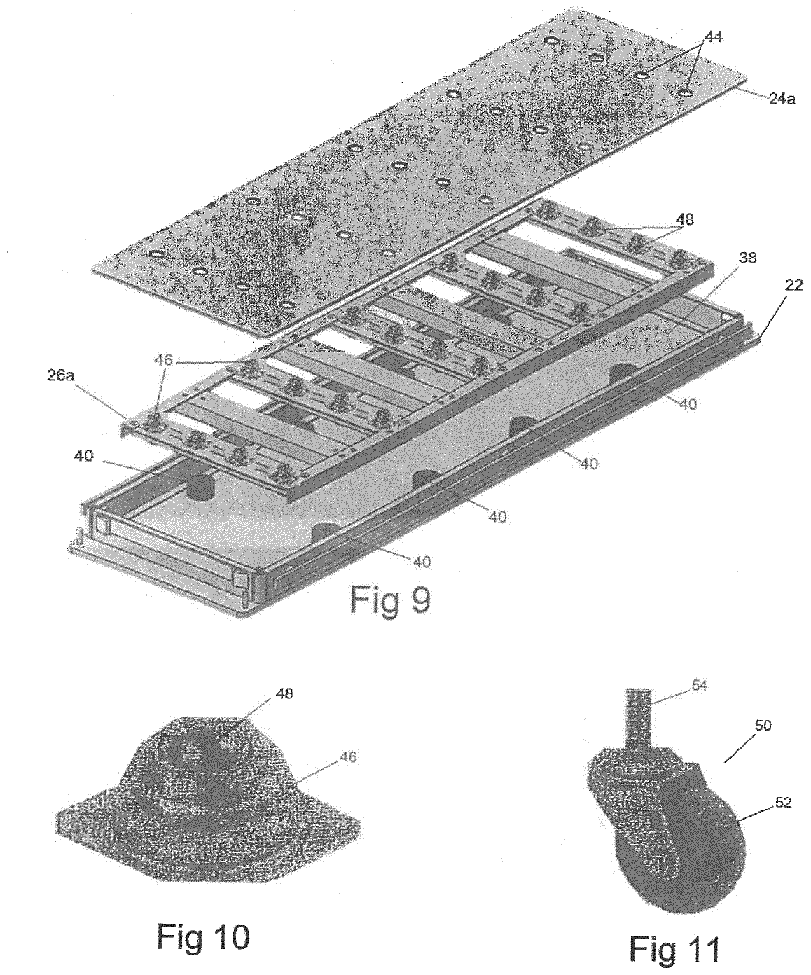

As shown in FIG. 9, importantly a second set of beams 34 mount and register a selected number of rollers or wheels 36 in position for entry into respective of the openings 30. When the lifting rack 26 is raised, the rollers or wheels 36 extend through the openings 30 and project, at least in part, above the support structure. Typically, a patient is supported on a pad or mat or like member atop the support and is slightly raised or pushed upwardly by the rollers and enabled to be rolled laterally from one side to the other side of the bed. As noted with the openings 30, depending on the application, the rollers 36 may be increased number, in diameter, and width, such as where the weight or height the patients to be raised above the support structure is indicative of a more robust roller wheel support.

In the embodiment shown, the bed frame 22 includes a plurality of laterally spaced linear spacer flanges 42 that extend upwardly from the frame 22 and into the interior in an amount sufficient to support and space the lifting rack 26 above the pneumatic actuator 40, or array of actuators, upon lowering retraction of the lifting rack from the support structure.

In a preferred arrangement, the means 28 for raising and lowering the lifting rack comprises an array of separate and spaced apart pneumatic actuators 40. Each of the actuators in the array thereof is connected in parallel to the pneumatic air source to operate simultaneously to move actuator pistons in and out and raise and lower the lifting rack 26 and associated rollers 36. In one preferred arrangement, the pneumatic actuator, or actuators 40, is fixedly connected to the bed frame 22 and the respective piston end, or ends, is fixedly connected to the lifting rack 26.

In one application, the pneumatic actuator 40 is what is referred to as a single acting air valve. In such actuator, a single inlet port receives pressurized air and a spring or bias member internally of the actuator presses the piston thereof in one direction against the air pressure.

In another arrangement, the pneumatic actuator 40 is what is referred to as a double acting air valve. In such actuator, a pair of air inlet ports seletively receive air from the pneumatic pump to force the piston thereof in opposite directions.

In another application, the pneumatic actuator, or array of actuators, is fixedly connected to the lifting rack and the respective piston end, or ends, extend to engage the bed frame.

The means 28 for raising and lowering the lifting rack 26 vertically upward and downward relative to the bed frame 22 comprises a selectively actuatable pneumatic air source, and at least one pneumatic actuator 40 in fluid connection to the air source. The pneumatic air source is not shown but is conventional and typically is located below the bed frame and may be hand operated or programmed to selectively provide pressurized air to the actuator.

The pneumatic actuator 40 includes a piston that provides a selective alternating in and out displacement movement upon selective actuation of the air source to move the lifting rack 26 and associated rollers 36 relative to the support structure 24 and the rollers relative to the opening 30 as desired. The pneumatic actuator 40 is connected to one of the lifting rack 26 and the bed frame 22 and the outward end of the piston thereof is adapted to engage the other of the lifting rack and the bed frame.

According to further embodiment of this invention, in FIG. 9 there is provided a medical table transport assembly 200, comprising: the bed frame 22; and a patient support structure or table 24A mounted atop the frame for supporting a person in a plane of the frame, the frame and support structure defining an interior chamber 38.

In this embodiment, the support structure or table 24 as modified as 24A to include an array of generally round openings 44.

The lifting rack 26 is modified as 26A and is characterized as a ball transfer table but as before is mounted in the chamber 38 for upward/downward vertical movement between the bed frame and the support structure. As shown in FIG. 8, transfer assemblies are each adapted to fit into respective of the spaces between respective of the spaced apart supports 22 formed by the sine wave. Each of a plurality of transfer beam 30 includes a series of wheels 32 and the wheels of the transfer beams are actuated simultaneously to move down and up relative to the respective transfer beam to which attached whereby to lift or lower the patient transfer member 12.

Referring to FIGS. 9 and 10, the ball transfer table 26A or modified lifting rack includes an array of rolling ball assemblies 46 with associated roller balls 48 sized for entry into respective of the openings 44, the roller balls 48 for supporting a person for movement, such as transfer or positioning atop the table when the lifting structure is raised and in the upward position and rotating to move the patient. The balls 48 may rotate 360 degrees for enabling positioning of a patient relative to the support table 24A.

As before, there is included the aforementioned means for raising and lowering the lifting rack vertically upward and downward relative to the bed frame, wherein the means for raising and lowering comprises a selectively actuatable pneumatic air source, and at least one pneumatic actuator in fluid connection to the air source, the actuator having a piston that provides a selective alternating in and out displacement movement upon selective actuation of the air source to move the lifting rack and associated rollers relative to the support structure and the rollers relative to the opening as desired, wherein the pneumatic actuator is connected to one of the lifting rack and the support structure and the outward end of the piston thereof is adapted to engage the other of the lifting rack and the support structure.

Referring to FIG. 8, an alternate preferred embodiment of a medical transfer system 34 according to this invention includes a patient support 36 in the form of a sheet of radiolucent fabric and a support, which support comprises a series of separate inflatable feet 38 of a suitable radiolucent material fixedly connected to the bottom surface of the sheet, the material being of minimal thickness for improved x-ray contrast. The same type of comb like toothed actuator 14 is used. FIG. 8 shows the feet 38 when inflated below the sheet 36 and FIG. 9 shows the feet 38 when not inflated.

The foregoing description of specific embodiments of the present invention have been presented for purposes of illustration and description. They are not intended to be exhaustive or to limit the invention to the precise form disclosed, and modifications or variations are possible in the light of the above teaching.

1. A medical bed and patient transfer system, comprising:
(a) a patient transfer table;
(b) a second table which is either a surgical table or an x-ray table;
(c) a patient support member transferable between the patient transfer member and the second table,
the patient support system including a sinusoidal structure and a mat disposed thereatop for supporting a patient thereon.

2. The system of claim 1, wherein the sinusoidal structure is formed from a sufficiently strong material to support a patient thereon.

3. The system of claim 2, wherein the material comprises a radiolucent material to enable an x-ray of the patient to be taken when on the x-ray table.

4. The system of claim 1, wherein: the patient transfer table includes a patient transfer member, the transfer member comprising:
(a) a patient support member having an upper surface for supporting a patient and a lower surface, and (b) a series of supports extending between the upper and lower surfaces fixedly connected to said lower surface for supporting the transfer system and said patient transport support and the second table, the supports in spaced apart relation between the opposite longitudinal ends of the surfaces, forming receiving spaces, and each support extending downwardly a like distance from the lower surface, the support member and supports comprising a radiolucent material and having a comb-like toothed apparatus disposed beneath a patient, the comb-like toothed apparatus extending transversely from an actuator bar in spaced apart relation.

5. The system of claim 4 which further comprises:
a plurality of transfer beams adapted to fit into the spaces formed between respective of the spaced apart supports, each transfer beam including a series of wheels that are actuated to simultaneously move down and up relative to the respective transfer beam whereby to lift or lower the patient from the second table to the transport member or transfer member and wherein when the wheels are raised, the transfer beams may enter or retract from the spaces formed by the comb-like toothed apparatus and when the transfer beams are lowered, the wheels raise the transfer member into contact with and lift the transfer member and the actuator bar allows the transfer member with a patient thereon to be rolled to the opposite patient surface.

6. The system of claim 5, wherein the spaced apart supports define successive portions of a sine wave, and further wherein successive upper peaks of the wave are connected to the lower surface and successive lower peaks of the wave form the supporting structure and which is movable between the second table and the transport table.

7. The system of claim 6, wherein the sine wave is formed from a radiolucent material that differs in vertical thickness across the patient support member such that a vertically directed x-ray through the transfer member and support structure sees the same total thickness of material everywhere.

8. The system of claim 7, wherein the spaced apart supports of a radiolucent material are formed as one integral unit that varies in thickness and uniformly attenuates electromagnetic waves passing therethrough.

9. The system of claim 7, wherein the vertical integral unit defines a sine wave with peaks and valleys, respectively, fixedly connected to the lower surface and adapted to form a support for the transfer system, the thickness of the vertical integral unit being such that the thickness of the support unit at the peak and valley is about 50% greater than the vertical thickness of the support unit therebetween and wherein the radiolucent material is selected from the group consisting of carbon fiber, polymethylmethacrylate (PMMA), PETG co-polyester and expanded polystyrene foam.

10. The system of claim 4, wherein the series of supports comprises a series of separate inflatable feet of radiolucent material fixedly connected to a bottom surface of a fabric, the fabric of minimal thickness for improved x-ray contrast.

11. The system of claim 4 wherein the series of supports comprises a series of separate and movable feet of a suitable radiolucent material connected to the bottom surface of the patient support, the feet location improving imaging contrast of x-rays through the patient support.

12. The system of claim 4 which further comprises: removable means for locking the comb-like toothed apparatus to the patient transfer member to enhance the toothed apparatus in moving the patient transfer member.

13. A medical table assembly comprising:
(a) a bed frame;
(b) a support structure mounted atop the frame for supporting a person in a plane of the frame, the frame and support defining an interior chamber, said support including an array of openings;
(c) a lifting rack mounted in the chamber for upward/downward vertical movement between the bed frame and the support structure, said lifting rack including an array of rollers for entry into respective of said openings, the rollers for supporting, lifting and moving a person when the lifting rack is raised and in the upward position; and
(d) means for raising and lowering the lifting rack vertically upward and downward relative to the bed frame.

14. The assembly of claim 13, wherein said means for raising and lowering comprises a selectively actuatable pneumatic air source, and at least one pneumatic actuator in fluid connection with the air source, the actuator having a piston that provides a selective alternating in and out displacement movement upon selective actuation of the air source to move the lifting rack and associated rollers relative to the support structure and the rollers relative to the opening, as desired.

15. The assembly of claim 14, wherein the pneumatic actuator is connected to one of the lifting rack and the support structure and the outward end of the piston thereof is adapted to engage the other of the lifting rack and the support structure, and further wherein the means for raising and lowering the lifting rack comprises an array of separate and spaced apart pneumatic actuators, each of the actuators in the array thereof connected in parallel to said air source to operate simultaneously to move the pistons in and out and raise and lower the lifting rack and associated rollers.

16. The system of claim 13, wherein said support structure includes an array of generally round openings, and the lifting rack comprises a ball transfer table that is mounted in the chamber for upward/downward vertical movement by the actuators between the bed frame and the support structure, the ball transfer table including a fixed array of ball assemblies that include a rolling ball sized for entry into respective of said round openings, the rolling balls for raising a person and rolling translating movement atop the support for patient positioning atop the support structure or movement between opposite ends, or in a side by side movement and transfer from the support table.
